(19) United States
(12) Patent Application Publication
(10) Pub. No.: US 2004/0166542 A1
(43) Pub. Date: Aug. 26, 2004

(54) Non-invasive urine test for potential prostate abnormalities

(76) Inventor: Hari Har Parshad Cohly, Brandon, MS (US)

Correspondence Address: COHLY, HARI HAR PARSHAD, 235 HOLMAR DRIVE, BRANDON, MS, 30947, US

(21) Appl. No.: 10/265,225

(22) Filed: Oct. 7, 2002

(51) Int. Cl. G01N 33/574

(52) U.S. Cl. 435/7.23

(57) ABSTRACT

Currently, serum is used more often than urine to detect prostate specific antigen (PSA). The need for a non-invasive test yielding similar results led us to develop a urine test that uses solar irradiated water as a reactant species. To develop this technology, seven reagents plus one control were produced by exposure of water for 40 days in sunlight to the colors of the visible spectrum through colored cellophane, control being an unwrapped bottle of sterile water. Serum PSA and the urine was tested using the above reagents. A positive urine test was observed with yellow-filtered irradiated water which absorbed at 454 nm. Positive results for the urine test had PSA levels of 0.21-4.0 ng/ml. Thus, this invention describes a non-invasive urine test mainly positive with PSA 0.21-4.0 ng/ml.

### BACKGROUND OF INVENTION

Prostate cancer is recognized as the most common type of cancer and the second leading cause of cancer-related deaths in men [1]. The principle screening tests for detection of asymptomatic prostate cancer are the digital rectal examination and measurement of the serum marker prostate specific antigen (PSA) [2]. Screening may also include a urine test to check for blood or infection.

The principal drawback of the PSA test is its imperfect specificity, owing to the fact that common conditions, such as benign prostate hyperplasia (BPH) and prostatitis, can cause borderline or even markedly abnormal test results [3, 4]. These results can lead to expensive diagnostic evaluation and unwarranted patient anxiety. At the other extreme, the high sensitivity of the test can result in over diagnosis. There is always the chance that small, indolent tumors which might require no treatment and which may never have surfaced clinically, would be gathered in the same net and indicated to be aggressive, potentially life threatening cancers [5]. There is also debate over the level of PSA-2.5 versus 4.0 ng/ml-that could be considered abnormal and might warrant biopsy [6, 7]. For this reason, an alternate urine test, which is partially positive with a serum PSA of <4.0 ng/ml, may be useful in detecting a subgroup of patients who do not yet require tumor biopsy.

The rational for choosing the current approach is based on the use of solar irradiated water for healing purposes some dating back to ancient healers and others to a more recent use of solarized water with certain additives [8, 9]. Our main interest was to develop a non-invasive in-vitro diagnostic test using a chemical reaction between solarized water and urine as reagents in a 96-well plate format.

Sun exposure for a minimum of five hours and maximum of 48 hours has been used to decontaminate water from bacteria and viruses [10-15]. Solarization for a minimum of six weeks has also been used to control pest infestation in farming [16]. The biomodulatory effects of individual colors of the spectrum have not been fully characterized.

Currently, serum is used more often than urine to detect prostate specific antigen (PSA). The need for a non-invasive test yielding similar results led us to develop an innovative urine testing that uses solar irradiated water as a reactant species. The invention pertains to the detection of a reactant in the urine which reacts with yellow filtered solar irradiated water.

Solarization of water has existed in traditional medicine and has been used to cure various ailments. The term “prostate specific antigen” was first introduced in the scientific literature by Richard Ablin and co-workers in the USA who published their work in the Journal of Reproduction and Fertility as early as in 1970 (17). This patent addresses the usefulness of irradiated water in determining the colorometric changes in the reaction between irradiated water and the urine from patients with prostate abnormality. For this purpose, the characteristics of the interaction between the urine and monochromatic irradiated waters were first compared with polychromatic irradiated water. Positive colorometric tests were compared with the distribution profile of serum PSA. The absorbency profile of a positive reaction was also investigated spectrophoretically for the first time.

### BRIEF SUMMARY OF THE INVENTION

To develop this technology, seven reagents plus one control were produced by exposure of water for 40 days in sunlight to the colors of the visible spectrum through colored cellophane, control being an unwrapped bottle of sterile water. Serum PSA and urine were tested using the above reagents. A positive urine test was observed with yellow-filtered irradiated water that absorbed at 454 nm. Thus, this invention describes a non-invasive urine test mainly positive with PSA within a certain range.

### REFERENCES

1. Varmus. H.; Klausner, R. Planning for Prostate Cancer Research. NIH Pamphlet, June, 1999.

2. Luboldt, H.; Rubben, H. PSA-based early detection of prostate cancer. Urologe—Ausgabe A 2000, 39, 22-26.

3. Di Silverio, F.; D'Eramo, G.; Buscarini, M.; Sciarra, A.; Casale, P.; Di Nicola, S.; Loreto, A. [The role of prostate specific antigen and its derivatives (age-specific PSA, PSA density, velocity, free/total PSA) in the diagnosis of prostate cancer] Minerva Urol. Nefrol. 1998, 50, 143-154.

4. Hofer, C.; Sauerstein, P.; Wolter, C.; Scholz, M.; Hartung, R.; Breul, J. Value of free prostate-specific antigen (Hybritech Tandem-R) in symptomatic patients consulting the urologist. Urol. Int. 2000, 64, 18-23.

5. Smith, R. A.; Mettlin, C. J.; Davis, K. J.; Eyre, H. Am. Cancer Soc. Guidelines for the Early Detection of Cancer. CA Cancer J. Clin. 2000, 50, 34-49.

6. Arcangeli, C. G.; Ornstein, D. K.; Keetch, D. W.; Andriole, G. L. Prostate-specific antigen as a screening test for prostate cancer. The United States experience. Urol. Clin. North Am. 1997, 24, 299-306.

7. Ornstein, D. K.; Pruthi, R. S. Prostate-specific antigen. Expert Opin. Pharmacother. 2000, 1, 1399-411.

8. Bickers, D. R., Pathak, M. A. Psoralen pharmacology: studies on metabolism and enzyme induction. National Cancer Institute Monographs 66,77-84 (1984).

9. Willard, J. W. Method of therapeutically treating damaged and/or infected tissue in warm blooded animals and compositions therefore, U.S. Pat. No. 3,984,540, October 5 (1976).

10. Accra, A.; Jurdi, M.; Mu'allem, H.; Karahagopian, Y.; Raffoul Z. Water Disinfection by Solar Radiation: Assessment and Application. International Development Research Center IDRC-TS66e, Ottawa, Ontario, Canada, 1990.

11. Wegelin, M.; Canonica, S.; Mechsner, K.; Fleischmann, T.; Pesaro, F.; Metzleet, A. Solar Water Disinfection: Scope of the process and analysis of radiation experiments. J. Water SRT-Aqua 1994, 43, 154-169.

12. Wegelin, M.; Sommer, B. Solar water disinfection (SODIS). Destined for Worldwide Use? Waterlines 1998, 16, 30-32.

13. McGuigan, K. G.; Joyce, T. M.; Conroy, R. M.; Gillespie, J. B.; Elmore-Meegan, M. Solar disinfection of drinking water contained in transparent plastic bottles: characterizing the bacterial inactivation process. J. Appl. Microbiol. 1998, 84, 1138-1148.

14. McGuigan, K. G.; Joyce, T. M.; Conroy, R. M. Solar disinfection using sunlight to decontaminate drinking water in developing countries. J. Med. Microbiol. 1999, 48, 758-787.

15. SODIS-News. Solar Water Disinfection. A Water Treatment Process used at Household Level. Available at: http://www.sodis.ch. Accessed Sep. 25, 2002.

16. Chellemi, D. O.; Olson, S. M.; Mitchell, D. J.; Secker, I.; McSorley, R. Adaptation of soil solarization to the integrated management of soilborne pests of tomato under humid conditions. Phytopathology 1997, 87, 250-258.

17. Ablin, R. J., Soanes W. A., Bronson P, Witebsky E. Precipitating antigens of the normal human prostate. J. Reprod. Fertil 1970, 3, 573-4.

### DETAILED DESCRIPTION OF THE INVENTION

Preparation of Reagent Water

Sterilized (double-distilled) water in sterilized translucent (50% transparent) plastic (a copolymer of 98% polypropylene and 2% polyethylene) bottles was obtained from Baxter (Deerfield, Ill.). The labels were removed from the bottles. The bottles were then wrapped in cellophane corresponding to the visible monochromatic spectral colors (violet, indigo, blue, green, yellow, orange, and red). Controls included one unwrapped bottle of sterilized water. The sterilized water in plastic bottles was incubated during the months of October and November in Jackson, Miss. The bottles (minimum of two bottles for each coloration were exposed to all daily hours of sunlight for 40 day and were then wrapped in aluminum foil without removing the cellophane and placed in the dark to avoid unwanted light exposure.

Urine Test Procedure

Urine was collected from patients who visited the urology clinic between the hours of 8.00 a.m. and 12.00 p.m. The first voided urine was collected and dip-test analysis of the urine was performed. Lack of infection was established. None of the subjects tested were under medication. The urine sample was stored at 4° C. until it could be processed. The pH of the urine samples was measured again before testing. The pH of the reagent water was 7.0. We tested urine samples of pH 6.0 to 6.8 to be within one pH unit difference. Urine samples (100 μl) taken in triplicates were mixed with 100 μl of different irradiated water samples and incubated at room temperature for seven days in a 96-well, flat bottomed tissue culture plate with a low evaporation lid. The first three wells were incubated with the control (polychromatic) irradiated water, followed by three wells of violet, three of indigo, and three of blue in the 1st row of the plate. The second row was incubated with four wells each of green, yellow, orange, and red irradiated waters. Thus, two rows of a 96-well plate were used for each patient's urine.

To further characterize the specificity of the reaction, 2 ml of urine was mixed with an equal volume of irradiated water and incubated in a test-tube for seven days at room temperature. The contents were then transferred to cuvettes to determine the absorbency profile at different wavelengths.

Healthy male (N=10; median age 33 years) and female volunteers (N=10; median age 36 years) were chosen from laboratory and hospital personnel to act as the control group. Normal reactions did not undergo serum PSA evaluation. The urine test using yellow irradiated water observed no detectable color reaction for both the male and female volunteers, except for one male. This individual, who had no disease symptoms, was followed up with a serum PSA test, which was determined as 3.3 ng/ml. This individual was excluded.

PSA Determinations

Serum PSA was measured with the Hybritech Tandem-R monoclonal radioimmunoassay. This assay was done by an independent urology lab. The results of the urine test were compared with the PSA test. The PSA distribution was arranged in the following categories: <0.1, 0.1-0.2, 0.214.0, 4.1-10.0, >10.0 (Table 1).

The average pH of the urine collected and tested was 6.0-6.8. The exposed water had an average pH of 7.0. We wanted to stay within 1 pH unit for the urine test so as to collect data that is not attributed to pH fluctuations. There were two males with PSA 0.21-4 ng/ml whose pH value did not lie between 6.0 and 6.8 that were excluded.

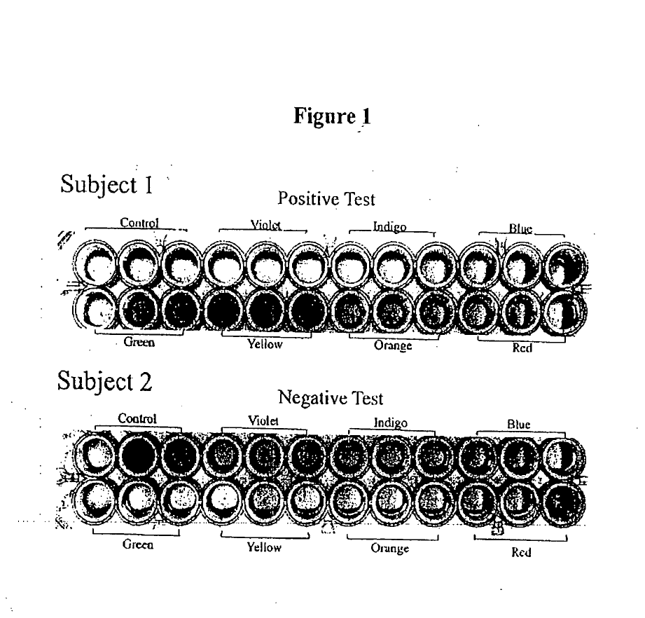

In FIG. 1, there is a clear-cut difference between the control (polychromatic irradiated) water and the yellow-filtered irradiated water. Two rows with a positive test marked subject 1 corresponding to a darkening of the pigmentation of the urine with yellow-irradiated water are shown. A negative test of the urine is shown in the lower portion of FIG. 1. There is some staining that occurs in other than yellow irradiated water as shown in the two plates, but the difference in staining between this and the yellow irradiated water is distinctly different. This test represents only a macroscopic test result as seen by the naked eye.

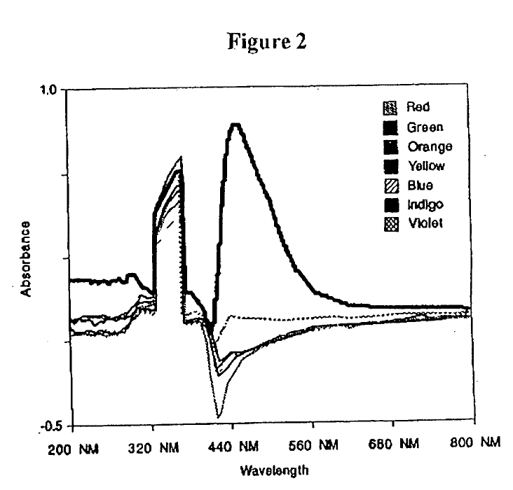

The observations made in the presence of the control-irradiated water were compared with water exposed to sunlight through different colored cellophane papers; i.e., the polychromatically irradiated water was used as a blank for the spectrophotometer reading. FIG. 2 is a representative distribution profile of a reaction between urine and different irradiated waters. There are two peaks and one valley in the absorbency profile. The peaks occur at 397 nm and 454 nm. The valley occurs at 418 nm. Peak at 454 nm corresponds to a reaction specifically between yellow-filtered irradiated water and urine.

The positive patient urine tests were then compared with their PSA readings. The results of the PSA readings were arbitrarily divided into five categories as follows: <0.1; 0.1-0.20; 0.21-4.0; 4.0-10.0; and >10.1 ng/ml. A positive urine test was observed in forty-five patients. The majority of positive patients (25 of 45) had a PSA level of 0.21-4.0 ng/ml. Twelve out of 45 had a PSA value<0.21, and six of 45 were above the 4.0-ng/ml level. There were 2 (out of 45) false-positive tests indicated by a negative PSA reading. In the same range of 0.21-4.0 ng/ml PSA, there were 27 out of 52 false-negative tests.

There was no reaction in urine of normal women (N=10; median age 36 yrs), nor in urine of normal males (N=9; median age 33 yrs) using the yellow-filtered irradiated water.

The serological determination of PSA is widely accepted to be the best method for screening, diagnosis, and follow-up in prostate cancer. PSA is not only present in the serum but also in other body fluids such as urine and semen specimens [16,17].

The results reported in this investigation used irradiated water as an alternative testing system. The first step in developing a urinary testing kit for the detection of prostate abnormality in our investigation was to determine if there was any unique reaction between the urine and the irradiated waters. A unique color reaction was observed when the urine reacted with yellow-filtered irradiated water. The fluid in the wells became dramatically deeper in yellow pigmentation. It is noteworthy that the irradiated water was not colored yellow, but was irradiated by sunlight through yellow colored cellophane for 40 days. Thus, the chemical change that took place is due solely to a reaction between the urine and the water irradiated at 565-575 nm. This change in coloration was considered to be a positive urine test. There is an inherent control in the design of this test, because all three wells gave a similar reaction. This reaction was further tested in the presence of urine from healthy females and healthy males whose results were negative. A positive urine test was observed in 45 patients; of that, 25 patients had a total PSA of 0.21-4.0 ng/ml.

In our lab studies, we have previously observed evidence of alterations in chemical and physical properties of water and biological functions by using colored cellophane during sun exposure (unpublished observations). Water exposed (E) to visible spectral emissions of sunlight was found to have an altered elemental composition, electrical conductance, osmolarity and salt-solubility. A difference in bio-modulatory effects was also observed. A gradual increase in leaching of Boron from E-violet to E-red was observed. The maximal increase in electrical conductance and maximal salt solubility of sodium bicarbonate was found with E-indigo. E-blue inhibited phyto-hemagglutinin-induced immune cell proliferation and inhibited mosquito larvae hatching, while E-orange stimulated root elongation in seed germination. A point to note is that solarization has been used to decontaminate drinking water which is based on the exposure of living organisms (bacteria and viruses) to UV-light and possible thermal inactivation. Sterile water has been exposed to sunlight irradiation for a long period of time (40 days), a procedure that clearly has a different mode of action compared to disinfection.

We have clearly shown that there is a chemical change in the presence of yellow-filtered irradiated water manifested by a change in the coloration of the urine with an absorbance peak at 454 nm. (Note: In the 96-well plate is that the absorbance peak was at 454 nm, while the color was macroscopically observed, i.e., the yellow pigmentation corresponds to the emission spectra in the yellow color range [565-575 nm]).

A visual change in the indicator system was considered optimum. The specifics of the changes in the calorimetric reaction were determined by evaluating the optical density in a spectrophotometer. It is clear that urine is carrying the causative agent for the color reaction while the irradiated water carries the antidote for this agent. A color reaction takes place because of the attraction or interaction of a component or components in the urine with the irradiated water. The mechanism of action between the yellow-filtered monochromatic irradiated water and the urine, however, is not completely understood. Some trace amounts of proteins, hormones and other substances normally found in urine may potentially interact with solarized water. It is not clear whether the color reaction observed was a simple reaction between the dye constituting the pigment in the urine or some combination of secreted protein and dye. It is possible that the sex hormones, like testosterone, may interact with the pyrrolic ring structure present in the pigment. This test predominantly detects a subset of patients whose PSA is in the range of 0.21-4.0 ng/ml.

Another important point to consider is the relationship of PSA in urine and serum. High levels of PSA in serum are suggestive of metastasis while PSA in the urine may indicate either the presence or absence of disease.

Through this study a new innovative test that has been created is easy to use, involves different biochemical assays than antibody mediated PSA tests, and uses urine instead of serum. Urine is more easily obtained and disposed of and less risky to handle than blood products. For the patient, the use of urine instead of blood eliminates the necessity of an invasive procedure and its possible complications. The ingredients of the kit, i.e., urine, yellow-solar-irradiated water, and a 96-well plate are environmentally friendly. The innovative technology is the transfer of solar energy into the bottled water. This technology is natural, simple, and has never been employed for in vitro diagnostic testing.

I present evidence here that a new urine test has been developed that is negative in women and healthy males but is positive in a subset of patients, especially in those patients with serum PSA 0.21-4.0 ng/ml.

TABLE 1
Distribution profile of the urine test with PSA measurements.

| PSA | (+) Urine | (−) Urine | Total |
|---|---|---|---|
| 0 | 2 | 6 | 8 |
| <0.1 | 5 | 19 | 24 |
| 0.1-0.2 | 7 | 17 | 24 |
| 0.21-4.0 | 25 | 27 | 52 |
| 4.0-10.0 | 2 | 6 | 8 |
| >10.0 | 4 | 7 | 11 |
| | 45 | 82 | 127 |

1. I claim that I have developed a new non-invasive urine test which specifically reacts with an agent in the urine of patients with a serum prostate specific antigen in the range of 0.21-4.0 ng/ml.

2. I claim that the aforementioned test uses yellow filtered irradiated water in comparison to polychromatic irradiated water.

3. I claim that the aforementioned test uses yellow-filtered irradiated water and polychromatic irradiated water incubated in the sun for a variable period of time.

4. I claim that I have developed a urine test that has an absorbance range 440-460 nm.

5. I claim that the yellow filtered-irradiated water in comparison to polychromatic irradiated water used for this test is the indicating reactant.

6. I claim that the yellow filtered-irradiated water in comparison to polychromatic irradiated water used for this test neutralizes the reactant species in the urine.
